(19) United States
(12) Patent Application Publication
(10) Pub. No.: US 2025/0115851 A1
(43) Pub. Date: Apr. 10, 2025

(54) CARTRIDGE, LIQUID TRANSFER SYSTEM, AND METHOD

(71) Applicants: CANON KABUSHIKI KAISHA, Tokyo (JP); Canon Medical Systems Corporation, Otawara-shi (JP)

(72) Inventors: Akane KUBOYAMA, Kawasaki (JP); Tetsuya SAKAI, Ryugasaki (JP); Kazutaka YANAGITA, Yokohama (JP); Yuhi HIGUCHI, Tokyo (JP)

(73) Assignees: CANON KABUSHIKI KAISHA, Tokyo (JP); Canon Medical Systems Corporation, Otawara-shi (JP)

(21) Appl. No.: 18/905,920

(22) Filed: Oct. 3, 2024

(30) Foreign Application Priority Data
Oct. 6, 2023 (JP) 2023-174700

(51) Int. Cl. C12M 1/00; C12M 1/34

(52) U.S. Cl. CPC C12M 29/14; C12M 29/04; C12M 41/40

(57) ABSTRACT

A cartridge used for cell preparation or cell processing includes a first container configured to store a liquid; an air filter arranged at an opening; a first passage connected to the first container and the opening and configured to allow the liquid suctioned from the first container to flow in; a second passage connected to the first passage and the opening and configured to allow the liquid to flow in from the first passage; and a second container connected to the second passage and configured to allow the liquid to flow in from the second passage if the first passage is pressurized with the liquid having flown into the first passage.

### CROSS-REFERENCE TO RELATED APPLICATIONS

This application is based upon and claims the benefit of priority from Japanese Patent Application No. 2023-174700, filed Oct. 6, 2023, the entire contents of which are incorporated herein by reference.

### FIELD

Embodiments described herein relate generally to a cartridge, a liquid transfer system, and a method.

### BACKGROUND

The preparation of an induced pluripotent stem cell (hereinafter referred to as an “iPS cell”) is performed manually by an operator with skills using equipment such as a safety cabinet. Thus, the preparation of an iPS cell requires considerable expense. This has been one of the obstacles to clinical application.

There are apparatuses such as a closed cell preparation apparatus that automates performance of predetermined processes such as cell culturing and a cell processing apparatus that allows a user to set any process. In order to realize such an apparatus at a low cost, the following configuration can be considered: Operating a closed cartridge incorporating a passage through which a liquid such as a cell suspension flows by using a liquid delivery driving mechanism such as a syringe pump or a peristaltic pump.

Using a syringe pump, however, requires equipment such as a safety cabinet since the sterile condition of the syringe cannot be secured. Using a peristaltic pump also requires an operator to be skilled when arranging a passage containing a reagent or a cell suspension in the pump. Even if a skilled operator installs a peristaltic pump, a problem such as detachment of a pipe may occur. Thus, a safe and inexpensive liquid transfer system is required.

### BRIEF DESCRIPTION OF THE DRAWINGS

FIG. 1 is a diagram showing a configuration of a liquid transfer system according to a first embodiment.

FIG. 2 is a diagram showing an example of a configuration of a cell handling mechanism according to the first embodiment.

FIG. 3 is a diagram showing an example of a configuration of a cartridge according to the first embodiment.

FIG. 4 is a flowchart illustrating a procedure of liquid transfer performed by the liquid transfer system according to the first embodiment.

FIG. 5 is a diagram showing a state in which a passage is depressurized by the liquid transfer system according to the first embodiment.

FIG. 6 is a diagram showing a state in which a passage is pressurized by the liquid transfer system according to the first embodiment.

FIG. 7 is a diagram showing an example of a configuration of a cartridge according to a second embodiment.

FIG. 8 is a diagram showing a state in which a passage is depressurized by the liquid transfer system according to the second embodiment.

FIG. 9 is a diagram showing an example of a configuration of a cartridge according to a first modification.

FIG. 10 is a diagram showing a state in which a passage is depressurized by the liquid transfer system according to the first modification.

FIG. 11 is a diagram showing an example of a configuration of a cartridge according to a second modification.

### DETAILED DESCRIPTION

In general, according to one embodiment, a cartridge used for cell preparation or cell processing includes a first container configured to store a liquid used for the cell preparation or the cell processing; an opening portion defining an opening opened toward outside; an air filter arranged at the opening and separating inside and outside of the cartridge so as to allow gas replacement; a first passage connected to the first container and the opening and configured to allow the liquid suctioned from the first container to flow in if the first passage is depressurized from the outside of the cartridge via the air filter; a second passage connected to the first passage and the opening and configured to allow the liquid to flow in from the first passage if the first passage is pressurized from the outside of the cartridge via the air filter with the liquid having flown into the first passage; and a second container connected to the second passage and configured to allow the liquid to flow in from the second passage if the first passage is pressurized with the liquid having flown into the first passage.

Hereinafter, a cartridge for cell preparation and a liquid transfer system using the cartridge will be described with reference to the accompanying drawings. In the description below, constituents having substantially the same functions and configurations will be denoted by the same reference symbols, and a repeat description of such constituents will be given only where necessary.

The description below introduces a case of using a cartridge and a liquid transfer system for handling an iPS cell as an example. Note that a cartridge and a liquid transfer system can be used not only to handle an iPS cell but also to handle any cells.

### First Embodiment

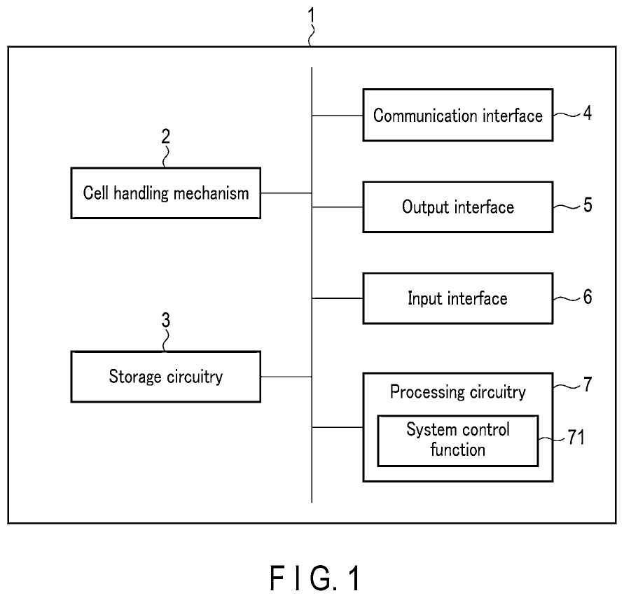

FIG. 1 is a block diagram showing an example of a configuration of a liquid transfer system 1 according to a first embodiment. The liquid transfer system 1 of the present embodiment is a cell handling system for automatically performing cell handling. The cell handling includes, for example, a process of preparing an iPS cell (hereinafter referred to as a “cell preparation process”) and/or a process of processing a cell (hereinafter referred to as a “cell processing process”). The cell preparation process or the cell processing process includes, for example, a process of separating blood cells in the blood to extract necessary cells, a process of introducing a factor into a peripheral blood mononuclear cell (hereinafter referred to as a “PBMC”) after expansion culture, a process of culturing a cell, etc. The liquid transfer system 1 may perform all or only some of the aforementioned processes. The liquid transfer system 1 may be connected to an external network or database via a network.

As shown in FIG. 1, the liquid transfer system 1 includes a cell handling mechanism 2, storage circuitry 3, a communication interface 4, an output interface 5, an input interface 6, and processing circuitry 7.

The cell handling mechanism 2 is a mechanism for performing cell handling. A detailed configuration of the cell handling mechanism 2 will be described later.

The storage circuitry 3 stores programs to be executed by the processing circuitry 7, various types of data to be used in the processing performed by the processing circuitry 7, and the like. Such programs include, for example, a program that is pre-installed in a computer via a network or a non-transitory computer-readable storage medium to cause the computer to implement each function of the processing circuitry 7. The various types of data as used herein are typically digital data. The storage circuitry 3 is an example of a storage.

The communication interface 4 is an interface for performing data communication with an external device via a network.

The output interface 5 is connected to the processing circuitry 7 and outputs a signal supplied from the processing circuitry 7. The output interface 5 is realized, for example, by display circuitry, print circuitry, a voice device, and the like. The display circuitry includes, for example, a CRT display, a liquid crystal display, an organic EL display, an LED display, a plasma display, and the like. The display circuitry may also be processing circuitry that converts data showing a display target into a video signal and outputs the video signal to an external device. The print circuitry includes, for example, a printer and the like. The print circuitry may also be output circuitry that outputs data showing a print target to an external device. The voice device includes, for example, a speaker and the like. The voice device may be output circuitry that outputs a voice signal to an external device.

The input interface 6 receives, for example, various operations from an operator. The input interface 6 is realized by, for example, a mouse, a keyboard, a touch pad which allows input of instructions through a touch on its operation screen, etc. The input interface 6 is connected to the processing circuitry 7, thereby converting an operational command that is input by the operator into an electric signal and outputting the electric signal to the processing circuitry 7.

The input interface 6 is not limited to one that includes physical operational components such as a mouse and a keyboard. For example, the input interface 6 may be processing circuitry that receives an electric signal corresponding to an operation command input from an external input device provided separately from the liquid transfer system 1 and outputs the electric signal to the processing circuitry 7.

The processing circuitry 7 is a processor that functions as the center of the liquid transfer system 1. The processing circuitry 7 performs a system control function 71 by executing a program read from the storage circuitry 3. With the system control function 71, the processing circuitry 7 comprehensively controls each component of the liquid transfer system 1. For example, in the system control function 71, the processing circuitry 7 controls the cell handling mechanism 2 so that the liquid transfer described later is performed. The processing circuitry 7 that implements the system control function 71 is an example of a controller.

The terminology “processor” used in the above description refers to, for example, circuitry such as a CPU (central processing unit), a GPU (graphics processing unit), an ASIC (application specific integrated circuit), a programmable logic device (such as a simple programmable logic device (SPLD), a complex programmable logic device (CPLD), and a field programmable gate array (FPGA)), etc. If the processor is, for example, a CPU, the processor implements the functions by reading and executing programs stored in the storage circuitry 3. On the other hand, if the processor is an ASIC, its functions are directly incorporated into the circuitry of the processor as logic circuitry, instead of being incorporated into a program being stored in the storage circuitry 3. Each processor of the present embodiment is not limited to being configured as single circuitry; multiple sets of independent circuitry may be integrated into a single processor that implements its functions. Furthermore, the plurality of components in FIG. 1 may be integrated into a single processor to implement the functions of the processor. The above description of the “processor” also applies to each of the embodiments and modifications described below.

In the present embodiment, descriptions will be given on the premise that each function is implemented by a single processor; however, the embodiment is not limited thereto. For example, a plurality of independent processors may be combined to constitute control circuitry, and the respective processors may implement the respective functions by executing the programs. The system control function 71 may be referred to as system control circuitry or installed as individual hardware circuitry. The above description of the respective functions implemented by the processing circuitry 7 also applies to each of the embodiments and modifications described below. The processing circuitry 7 may include a storage area for storing at least part of the data stored in the storage circuitry 3.

Next, a configuration of the cell handling mechanism 2 will be described in detail.

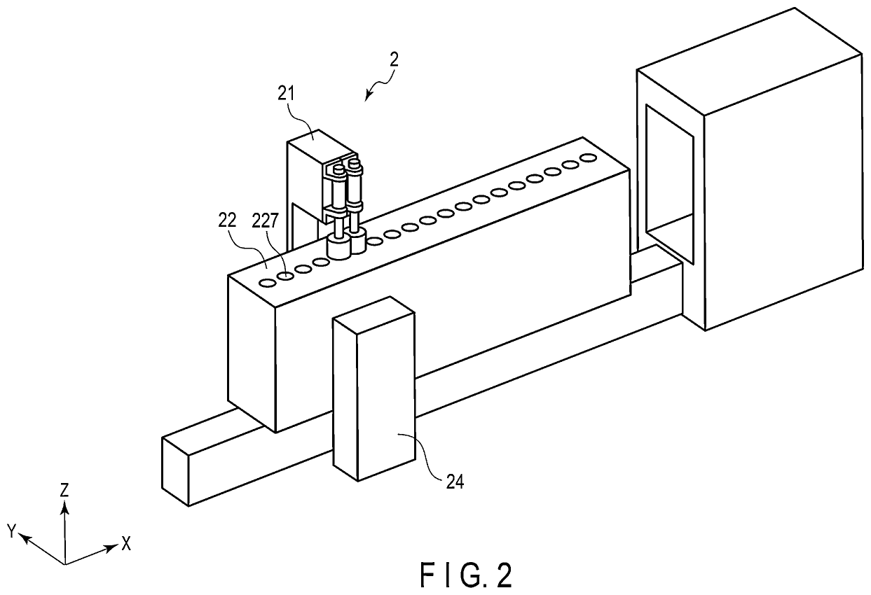

FIG. 2 is a diagram showing an example of a configuration of the cell handling mechanism 2. The cell handling mechanism 2 is a device for performing cell handling using a cartridge 22 for cell handling. As shown in FIG. 2, the cell handling mechanism 2 includes a liquid delivery mechanism 21 and a valve open-close mechanism 24. The liquid transfer system 1 is constituted by the cartridge 22 and a body apparatus including components other than the cartridge 22. The body apparatus is an example of a cell handling apparatus.

The cartridge 22 is a closed cartridge for automatically performing cell handling. The cartridge 22 performs one or more processes among the processes included in the cell preparation process or the cell processing process. The cartridge 22 is attached in a detachable fashion, and multiple types of cartridges 22 that perform different processes can be replaced with each other and can be attached to be used. To prevent infection, the cartridge 22 is discarded after use. Multiple cartridges 22 may be attached to the cell handling mechanism 2.

The cartridge 22 includes one or more closed passages and containers in the cartridge 22. Typically, the cartridge 22 includes multiple passages and multiple containers in the cartridge 22. Each passage is a passage for transporting a liquid including a reagent or a cell, and is formed of a tube-shaped resin or plastic. Each container is formed of a resin or plastic. For example, polycarbonate (PC) or polystyrene (PS) is used as a resin.

The cartridge 22 includes one or more liquid delivery units in the cartridge 22. Each liquid delivery unit includes an upstream-side container, a downstream-side container, and a passage connecting the upstream-side container with the downstream-side container. Each liquid delivery unit is used for a process of transferring a liquid contained in the upstream-side container to the downstream-side container (hereinafter referred to as a “liquid transfer process”) in the cell preparation process or the cell processing process. The liquid transfer process includes, for example, a process of transferring a liquid collected after the processing is performed to the next processing position, a process of transferring a cell liquid to pass the cell liquid through a filter, a process of transferring a liquid to an ejection port to diffuse the liquid from the ejection port, and the like. The liquid to be transferred may be blood, a blood suspension, a liquid containing PBMCs, a liquid containing cells such as a culture liquid, or a liquid used for cell processing such as a reagent, medium, or factor.

The liquid delivery mechanism 21 is connected to the cartridge 22. The liquid delivery mechanism 21 pressure-feeds a liquid inside the cartridge 22 by controlling the air pressure inside the cartridge 22 under the control of the processing circuitry 7. The liquid delivery mechanism 21 is, for example, a pneumatic pressure device having a compressor and/or a pressure pad. The liquid delivery mechanism 21 is an example of a pneumatic pressure control device.

Under the control of the processing circuitry 7, the valve open-close mechanism 24 opens and closes the valve (described later) in the cartridge 22 and switches the passages in the cartridge 22. The valve open-close mechanism 24 is an example of a passage switcher. The valve open-close mechanism 24 opens and closes the valve in the cartridge 22 without touching it by, for example, using a magnet.

Next, a configuration of the liquid delivery unit in the cartridge 22 will be detailed.

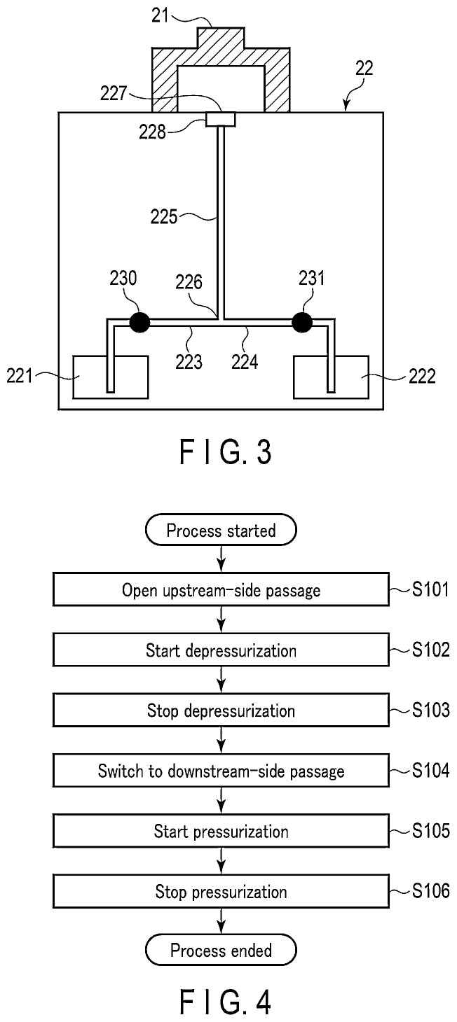

FIG. 3 is a diagram showing an example of a configuration of the cartridge 22. FIG. 3 shows one of the liquid delivery units in the cartridge 22. As shown in FIG. 3, the liquid delivery unit of the cartridge 22 includes an upstream-side container 221, a downstream-side container 222, an upstream-side duct line 223, a downstream-side duct line 224, a connection duct line 225, a connection portion 226, an opening portion 227, an air filter 228, an upstream-side valve 230, and a downstream-side valve 231. The upstream-side container 221, downstream-side container 222, upstream-side duct line 223, downstream-side duct line 224, connection duct line 225, connection portion 226, upstream-side valve 230, and downstream-side valve 231 are in the cartridge 22.

The upstream-side container 221 is a container for containing a liquid to be transferred and formed of a resin or plastic. The upstream-side container 221 is, for example, a reagent container, a blood bag, or a culture container. The upstream-side container 221 is an example of a first container.

The downstream-side container 222 is a container for containing a liquid transferred from the upstream-side container 221 and formed of a resin or plastic. The downstream-side container 222 is, for example, a culture container, a collecting container, or a waste liquid container. The downstream-side container 222 is an example of a second container.

For example, if the upstream-side container 221 is a reagent container, then a culture container, a collecting container, or a waste liquid container is used as the downstream-side container 222. In this case, a liquid to be transferred is a reagent. If the upstream-side container 221 is a blood bag, then for example a collecting container that includes a filter for separating blood cells is used as the downstream-side container 222. In this case, a liquid to be transferred is blood. If the upstream-side container 221 is a culture container, then a collecting container or a waste liquid container is used as the downstream-side container 222. In this case, a liquid to be transferred is a culture liquid.

A combination of the upstream-side container 221, the downstream-side container 222, and the liquid is not limited to the above combinations, and can be applied to all the processes for transferring a liquid in the cartridge 22 in the cell preparation process or the cell processing process. Also, a downstream-side container of a liquid delivery unit used in a process may be used as an upstream-side container of a transfer unit used in the next process.

The upstream-side duct line 223, the downstream-side duct line 224, and the connection duct line 225 are duct lines constituting respective passages and formed of a tube-shaped resin or plastic.

One end of the upstream-side duct line 223 is connected to the upstream-side container 221. At the connection portion 226, the other end of the upstream-side duct line 223 is connected to the downstream-side duct line 224 and the connection duct line 225. One end of the downstream-side duct line 224 is connected to the downstream-side container 222. At the connection portion 226, the other end of the downstream-side duct line 224 is connected to the upstream-side duct line 223 and the connection duct line 225. One end of the connection duct line 225 is connected to the opening portion 227. At the connection portion 226, the other end of the connection duct line 225 is connected to the upstream-side duct line 223 and the downstream-side duct line 224.

The upstream-side duct line 223 and the connection duct line 225 form an upstream-side passage connecting the upstream-side container 221 with the opening portion 227. The upstream-side passage is an example of a first passage. One end of the downstream-side duct line 224 is connected to the upstream-side passage, and the other end of the downstream-side duct line 224 is connected to the downstream-side container 222. The downstream-side duct line 224 branches off from the upstream-side passage (223, 225) and forms a downstream-side passage connecting the downstream-side container 222 with the opening portion 227. The downstream-side passage is an example of a second passage. The connection portion 226 connects the upstream-side passage (223, 225) with the downstream-side passage (224).

In the description below, only the downstream-side duct line 224 branching off from the upstream-side passage (223, 225) is referred to as a downstream-side passage (a second passage); however, a passage formed by the downstream-side duct line 224 and the connection duct line 225 may be referred to as a downstream-side passage (a second passage). In this case, the downstream-side passage (second passage) includes a part of the upstream-side passage (223, 225) and connects the downstream-side container 222 with the opening portion 227.

The opening portion 227 is at an end of the upstream-side passage (223, 225) and defines an opening opened toward the outside of the cartridge 22. A pressure pad of the liquid delivery mechanism 21 is attached to the opening portion 227.

The air filter 228 is arranged at the opening of the opening portion 227. The air filter 228 is a filter for separating the inside and the outside of the cartridge 22 so as to allow gas replacement. As the air filter 228, for example, a sheet-shaped sterile filter such as UE025020-25 manufactured by Saint-Gobain can be used as appropriate. With the air filter 228 provided, it is possible to suppress the outflow of the liquid inside the cartridge 22 to the outside and the inflow of bacteria into the cartridge 22 from the outside.

The upstream-side valve 230 and the downstream-side valve 231 are passage switchers for switching the upstream-side passage (223, 225) and the downstream-side passage (224). The upstream-side valve 230 is arranged at the upstream-side duct line 223 in the upstream-side passage (223, 225). The downstream-side valve 231 is arranged at the downstream-side duct line 224 in the downstream-side passage. The open-close states of the upstream-side valve 230 and the downstream-side valve 231 are switched by the valve open-close mechanism 24. With the upstream-side valve 230 opened and the downstream-side valve 231 closed, the upstream-side passage (223, 225) is opened, and the upstream-side container 221 and the opening of the opening portion 227 communicate with each other. With the upstream-side valve 230 closed and the downstream-side valve 231 opened, the downstream-side passage (224) is opened, and the downstream-side container 222 and the opening of the opening portion 227 communicate with each other.

Next, an operation of the liquid transfer system 1 of the present embodiment will be described.

FIG. 4 is a flowchart showing an example of a procedure of liquid transfer performed by the liquid transfer system 1. The liquid transfer is performed by the processing circuitry 7 when the liquid transfer process is performed. The liquid transfer process is, for example, one of the series of processes for performing cell handling. The procedure of each step described below is a mere example, and each step can be altered as appropriate to the extent possible. Omission, replacement, and addition of a step can be made as appropriate in the procedure explained below according to the manner in which the embodiment will be implemented. The foregoing explanation of the procedure of each step applies to each of the embodiments and modifications described below.

### (Step S101)

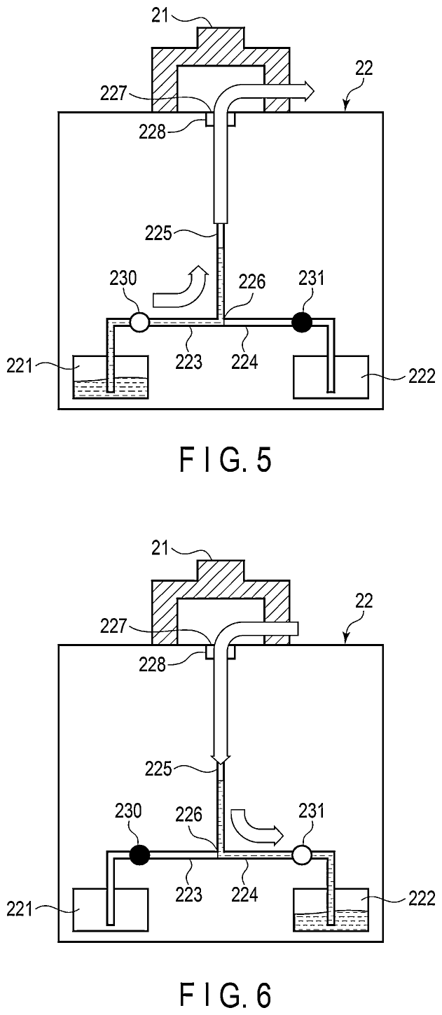

In the liquid transfer, the processing circuitry 7 first opens the upstream-side passage (223, 225), as shown in FIG. 5. At this time, the processing circuitry 7 keeps the upstream-side valve 230 opened and the downstream-side valve 231 closed by controlling the valve open-close mechanism 24. Thus, the upstream-side container 221 and the opening portion 227 communicate with each other, resulting in the upstream-side passage (223, 225) being open. Also, the downstream-side container 222 and the upstream-side passage (223, 225) are disconnected from each other, resulting in the downstream-side passage (224) being closed. In each of the drawings, the state in which the upstream-side valve 230 and the downstream-side valve 231 are opened is indicated by a white circle, and the state in which they are closed is indicated by a black circle.

### (Step S102)

Next, the processing circuitry 7 depressurizes the upstream-side passage (223, 225) via the opening portion 227 by controlling the liquid delivery mechanism 21, as shown in FIG. 5. When the upstream-side passage (223, 225) is depressurized, a liquid contained in the upstream-side container 221 is suctioned and flows into the upstream-side passage (223, 225).

### (Step S103)

When a predetermined period of time has elapsed since the start of the depressurization, the processing circuitry 7 stops the depressurization of the upstream-side passage (223, 225). The liquid that has flown into the upstream-side passage (223, 225) is stored in the upstream-side passage (223, 225).

### (Step S104)

Next, the processing circuitry 7 switches a passage to be used to the downstream-side passage (224). At this time, the processing circuitry 7 keeps the upstream-side valve 230 closed and the downstream-side valve 231 opened. Thus, the upstream-side container 221 and the opening portion 227 are disconnected from each other, resulting in the upstream-side passage (223, 225) being closed. Also, the downstream-side container 222 and the upstream-side passage (223, 225) communicate with each other, resulting in the downstream-side passage (224) being open.

### (Step S105)

Next, the processing circuitry 7 pressurizes the downstream-side passage (224) via the opening portion 227 and the connection duct line 225 by controlling the liquid delivery mechanism 21, as shown in FIG. 6. When the downstream-side passage (224) is pressurized, a liquid stored in the upstream-side duct line 223 or the connection duct line 225 flows in the downstream-side duct line 224, and the liquid that has passed through the downstream-side duct line 224 is discharged to the downstream-side container 222. That is, the liquid stored in the upstream-side passage (223, 225) flows in the downstream-side container 222 through the downstream-side passage (224).

### (Step S106)

When a predetermined period of time has elapsed since the start of the pressurization, the processing circuitry 7 stops the pressurization of the connection duct line 225.

Through above steps S101 to S106 being performed, the liquid stored in the upstream-side container 221 is transferred to the downstream-side container 222.

Hereinafter, the effects of the liquid transfer system 1 according to the present embodiment will be described.

The liquid transfer system 1 of the present embodiment includes the cartridge 22 used for cell preparation or cell processing and the processing circuitry 7. The cartridge 22 includes the opening portion 227, the air filter 228, the upstream-side container 221, the upstream-side passage (223, 225), the downstream-side passage (224), and the downstream-side container 222. The opening portion 227 defines an opening opened toward outside. The air filter 228 is at the opening of the opening portion 227 and separates the inside and the outside of the cartridge 22 so as to allow gas replacement. The upstream-side container 221 stores a liquid used for cell preparation or cell processing. The upstream-side container 221 is an example of a first container. The upstream-side passage (223, 225) is a passage connecting the upstream-side container 221 with the opening of the opening portion 227. If the upstream-side passage (223, 225) is depressurized from the outside of the cartridge 22 via the air filter 228, a liquid suctioned from the inside of the upstream-side container 221 flows in the upstream-side passage (223, 225). The upstream-side passage (223, 225) is an example of a first passage. The downstream-side passage (224) is a passage connecting the upstream-side passage (223, 225) with the downstream-side container 222. If the upstream-side passage (223, 225) is pressurized from the outside of the cartridge 22 via the air filter 228 with a liquid having flown into the upstream-side passage (223, 225), the liquid flows from the upstream-side passage (223, 225) into the downstream-side passage (224). The downstream-side passage (224) is an example of a second passage. If the upstream-side passage (223, 225) is pressurized with a liquid having flown into the upstream-side passage (223, 225), the liquid flows from the downstream-side passage (224) into the downstream-side container 222. The downstream-side container 222 is an example of a second container.

The upstream-side container 221 is, for example, a reagent container, a blood bag, or a culture container. The downstream-side container 222 is, for example, a culture container, a collecting container, or a waste liquid container. A combination of the upstream-side container 221 and the downstream-side container 222 is not limited to the above combinations, and combinations of all the containers used when transferring a liquid in the cartridge 22 can be applied.

Also, the cartridge 22 includes the upstream-side valve 230 and the downstream-side valve 231. The upstream-side valve 230 and the downstream-side valve 231 are examples of a passage switcher for switching the connection state of the upstream-side container 221 and the upstream-side passage (223, 225) and the connection state of the downstream-side passage (224) and the downstream-side container 222.

By controlling the liquid delivery mechanism 21, the processing circuitry 7 causes a liquid to flow from the inside of the upstream-side container 221 into the upstream-side passage (223, 225) by depressurizing the upstream-side passage (223, 225) and causes the liquid that has flown into the upstream-side passage (223, 225) to flow in the downstream-side container 222 through the downstream-side passage (224). At this time, by controlling the upstream-side valve 230 and the downstream-side valve 231 based on the fact that a liquid has flown from the inside of the upstream-side container 221 into the upstream-side passage (223, 225), the processing circuitry 7 switches the connection state of the upstream-side container 221 and the upstream-side passage (223, 225) from a connected state to a disconnected state, and switches the connection state of the downstream-side passage (224) and the downstream-side container 222 from a disconnected state to a connected state. The processing circuitry 7 functions as a pneumatic pressure controller for controlling the pneumatic pressure of the passage inside the cartridge 22 and a switching controller for switching the upstream-side passage (223, 225) and the downstream-side passage (224).

With the above configuration, the liquid transfer system 1 of the present embodiment can prepare an iPS cell or process a cell while maintaining a sterile condition by using the closed cartridge 22.

Also, the liquid transfer system 1 of the present embodiment includes the liquid delivery mechanism 21. With the use of the liquid delivery mechanism 21, after sucking a liquid up from the upstream-side container 221 by applying a negative pressure to the passage inside the cartridge 22, it is possible to move the sucked up liquid to the downstream-side container 222 by applying a positive pressure to the passage inside the cartridge 22.

Also, to prevent infection, the cartridge 22 is discarded after use. In the liquid transfer system 1 of the present embodiment, the liquid delivery mechanism 21 is arranged outside the cartridge 22; thus, the same liquid delivery mechanism 21 can be used even if the cartridge 22 is replaced, allowing for reduction in the costs of producing the liquid transfer system 1.

The opening portion 227 connected to the liquid delivery mechanism 21 is provided with the air filter 228. With the air filter 228, the inside of the cartridge 22 can be maintained in a sterile condition. For example, if a syringe is used instead of the liquid delivery mechanism 21 as a driving mechanism for transferring a liquid inside the cartridge 22, a sterile condition cannot be secured, and a safety cabinet thus needs to be used. The liquid transfer system 1 of the present embodiment can secure a sterile condition without using a safety cabinet.

Also, in the case of using a peristaltic pump as a liquid delivery mechanism, installing a pump requires the skill of the operator, and there is also a risk of breakdown such as detachment of a pipe. The present embodiment has a low risk of breakdown, as compared to the case of using a peristaltic pump, and liquid delivery can be performed safely by using the liquid delivery mechanism 21 that adopts a pressure-feeding method that allows for easy attachment and detachment.

In this manner, connecting the cartridge 22 with the liquid delivery mechanism 21 via the air filter 228 and transferring a liquid inside the cartridge 22 in an air pressure-feeding method that allows for easy attachment and detachment can maintain the inside of the cartridge 22 in a sterile condition, supply, from the outside, a driving force for moving the liquid inside the cartridge 22, and secure safety. That is, a mechanism for delivering a liquid inside the closed cartridge 22 can be realized with a simple structure, safely, and at a low cost.

### Second Embodiment

A second embodiment will be described. The present embodiment is a modification of the configuration of the first embodiment, as described below. Descriptions of the configuration, operation, and effect that are the same as those of the first embodiment will be omitted.

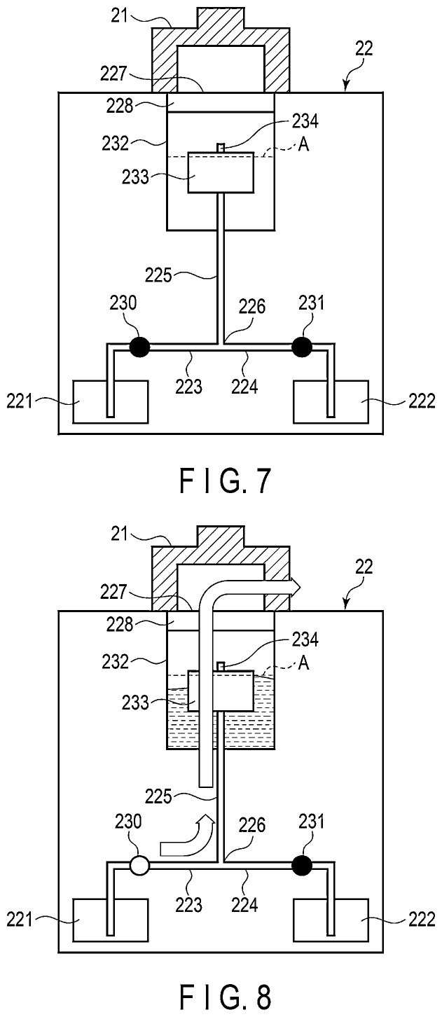

FIG. 7 is a diagram showing an example of a configuration of the cartridge 22 according to the present embodiment. FIG. 7 shows an example of a configuration of one of the liquid delivery units in the cartridge 22. As shown in FIG. 7, each liquid delivery unit of the cartridge 22 further includes a pressure chamber 232, and a disk filter 233 inside the cartridge 22 with respect to the air filter 228. The present embodiment adopts a double structure using two air filters that are the air filter 228 and the disk filter 233, thus being able to prevent contamination from the outside and maintain the safety in two stages. The disk filter 233 is an example of a second air filter.

The pressure chamber 232 is arranged between the opening portion 227 and the connection duct line 225 and connected to both the opening portion 227 and the connection duct line 225. The pressure chamber 232 is connected to the pressure pad of the liquid delivery mechanism 21 via the opening portion 227. The pressure chamber 232 is a space for receiving pneumatic pressure control by the liquid delivery mechanism 21 and has a diameter larger than the diameters of the duct lines 223 to 225. The pressure chamber 232 is formed of, for example, a resin or plastic. The liquid delivery mechanism 21 pressurizes or depressurizes the inside of the cartridge 22 by pressurizing or depressurizing the pressure chamber 232.

Also, the pressure chamber 232 includes a connection port 234. The connection port 234 is connected to the connection duct line 225 and opens to the inside of the pressure chamber 232. The pressure chamber 232 and the connection port 234 are paths through which a gas controlled by the liquid delivery mechanism 21 passes.

The disk filter 233 is arranged at the connection port 234 inside the pressure chamber 232. The disk filter 233 is, for example, an air filter for separating the pressure chamber 232 from the connection duct line 225 so as to allow gas replacement. For example, a disk-shaped sterile filter such as AP4954 manufactured by PALL Corporation can be used as the disk filter 233.

The pressure chamber 232 is used as a space for temporarily storing a liquid that has unexpectedly flown in. FIG. 8 shows the state in which the amount of liquid suctioned from the upstream-side container 221 to the upstream-side passage (223, 225) unintendedly exceeds a defined amount. Even if a liquid is excessively suctioned from the upstream-side container 221, the excessively suctioned liquid is temporarily stored inside the pressure chamber 232, as shown in FIG. 8, thus preventing the suctioned liquid from flowing out to the outside of the cartridge 22 through the opening of the opening portion 227.

Also, the pressure chamber 232 has a volume larger than the amount of liquid used. For example, the pressure chamber 232 is formed to have a volume larger than the volume of the liquid stored in the upstream-side container 221. Alternatively, the volume of the pressure chamber 232 may be larger than the volume of the upstream-side container 221. In this case, even if all the liquid stored in the upstream-side container 221 is suctioned, the excessively suctioned liquid is stored in the pressure chamber 232, and can thus be prevented from flowing out to the outside of the cartridge 22.

Also, as shown in FIG. 8, the connection port 234 is arranged above the position of the liquid surface A in the case where all the liquid stored in the upstream-side container 221 has flown into the pressure chamber 232. That is, the exit (connection port 234) of the air path (connection duct line 225) communicating with the pressure chamber 232 is positioned above the liquid surface A observed when all the amount of liquid has flown into the pressure chamber 232. With this structure, even if the excessively suctioned liquid is stored in the pressure chamber 232, it is possible to prevent the liquid from flowing back into the connection duct line 225 through the connection port 234 and also to prevent the liquid from flowing out to the outside through the air filter 228.

### First Modification

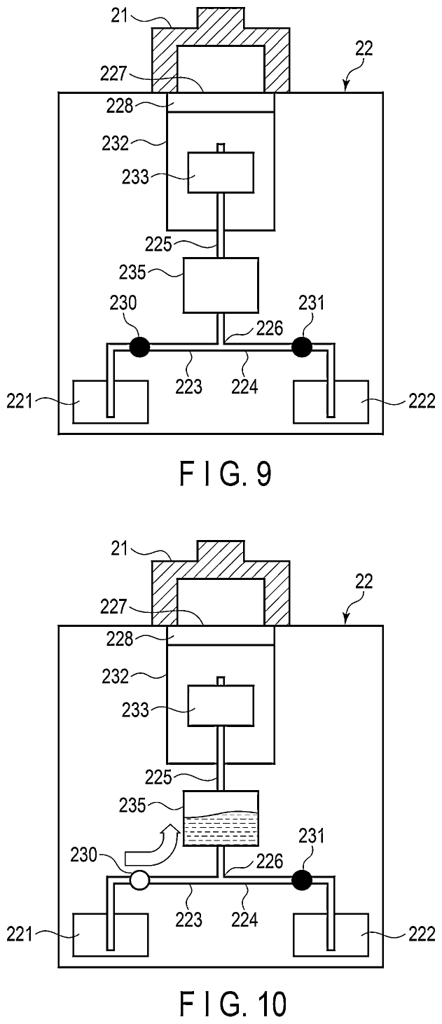

FIG. 9 is a diagram showing an example of a configuration of the cartridge 22 according to a first modification. FIG. 9 shows an example of a configuration of one of the liquid delivery units in the cartridge 22. As shown in FIG. 9, each liquid delivery unit of the cartridge 22 according to the present modification further includes a liquid storing container 235.

The liquid storing container 235 is arranged in the middle of the connection duct line 225. The liquid storing container 235 is a container for temporarily retaining a liquid suctioned from the upstream-side container 221, and has a diameter larger than the diameters of the duct lines 223 to 225. The liquid storing container 235 is formed of, for example, a resin or plastic. The liquid storing container 235 is an example of a storage.

FIG. 10 shows the state in which a liquid has been suctioned from the upstream-side container 221. When a liquid suctioned from the upstream-side container 221 is to be transferred, the movement of the liquid is easily controlled if the liquid to be controlled is stored all in one place. In the present modification, arranging the liquid storing container 235 for storing a liquid suctioned from the upstream-side container 221 in one place can improve the accuracy of the control performed when transferring the liquid suctioned from the upstream-side container 221.

### Second Modification

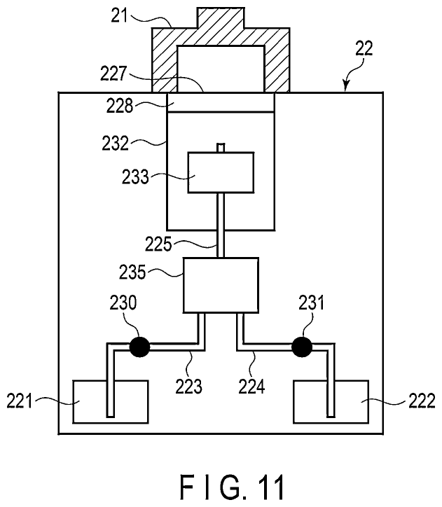

FIG. 11 is a diagram showing an example of a configuration of the cartridge 22 according to a second modification. FIG. 11 shows an example of a configuration of one of the liquid delivery units in the cartridge 22. As shown in FIG. 11, the liquid storing container 235 may be arranged instead of the connection portion 226. In this case, the upstream-side duct line 223, the downstream-side duct line 224, and the connection duct line 225 are connected to the liquid storing container 235.

### Other Modifications

A check valve may be used instead of the upstream-side valve 230 and the downstream-side valve 231. In this case, it is unnecessary for the processing circuitry 7 to perform control to switch passages. The check valve arranged in the middle of the upstream-side duct line 223 in place of the upstream-side valve 230 allows a liquid going from the upstream-side container 221 side toward the connection duct line 225 side to pass through but does not allow a liquid going from the connection duct line 225 side toward the upstream-side container 221 side to pass through. The check valve arranged in the middle of the downstream-side duct line 224 in place of the downstream-side valve 231 allows a liquid going from the downstream-side container 222 toward the connection duct line 225 to pass through but does not allow a liquid going from the connection duct line 225 toward the downstream-side container 222 to pass through.

Also, the operation of the liquid delivery mechanism 21 may be performed under the control of the processing circuitry 7 or performed through an operation by an operator.

According to at least one embodiment described above, a liquid in a closed cartridge can be transferred safely and at a low cost.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cartridge used for cell preparation or cell processing, the cartridge comprising:
a first container configured to store a liquid used for the cell preparation or the cell processing;
an opening portion defining an opening opened toward outside;
an air filter arranged at the opening and separating inside and outside of the cartridge so as to allow gas replacement;
a first passage connected to the first container and the opening and configured to allow the liquid suctioned from the first container to flow in if the first passage is depressurized from the outside of the cartridge via the air filter;
a second passage connected to the first passage and the opening and configured to allow the liquid to flow in from the first passage if the first passage is pressurized from the outside of the cartridge via the air filter with the liquid having flown into the first passage; and
a second container connected to the second passage and configured to allow the liquid to flow in from the second passage if the first passage is pressurized with the liquid having flown into the first passage.

2. The cartridge according to claim 1, further comprising a passage switcher configured to switch a connection state of the first container with the first passage and a connection state of the second passage with the second container.

3. The cartridge according to claim 1, further comprising a second air filter inside the cartridge with respect to the air filter.

4. The cartridge according to claim 1, further comprising a pressure chamber configured to receive depressurization and pressurization from the outside of the cartridge.

5. The cartridge according to claim 4, wherein the pressure chamber has a volume larger than a possible total amount of the liquid used for the cell preparation or the cell processing.

6. The cartridge according to claim 4, wherein
the pressure chamber includes a connection port communicating with the first passage, and
the connection port is positioned above a liquid surface of the liquid, the liquid surface of the liquid being observed in a state where all the liquid stored in the first container is stored in the pressure chamber.

7. The cartridge according to claim 1, further comprising a storage provided for the first passage and configured to store the liquid that has flown into the first passage.

8. The cartridge according to claim 1, wherein
the first container is a reagent container, a blood bag, or a culture container, and
the second container is a culture container, a collecting container, or a waste liquid container.

9. A liquid transfer system comprising a cartridge used for cell preparation or cell processing, and a pneumatic pressure control device, wherein the cartridge comprising:
a first container configured to store a liquid used for the cell preparation or the cell processing;
an opening portion defining an opening opened toward outside;
an air filter arranged at the opening and separating inside and outside of the cartridge so as to allow gas replacement;
a first passage connected to the first container and the opening and configured to allow the liquid suctioned from the first container to flow in if the first passage is depressurized from the outside of the cartridge via the air filter;
a second passage connected to the first passage and the opening and configured to allow the liquid to flow in from the first passage if the first passage is pressurized from the outside of the cartridge via the air filter with the liquid having flown into the first passage; and
a second container connected to the second passage and configured to allow the liquid to flow in from the second passage if the first passage is pressurized with the liquid having flown into the first passage,
and wherein the pneumatic pressure control device is configured to allow the liquid to flow into the first passage from inside of the first container by depressurizing the first passage and allow the liquid that has flown into the first passage to flow into the second container through the second passage by pressurizing the first passage.

10. The liquid transfer system according to claim 9, further comprising processing circuitry configured to control driving of the pneumatic pressure control device.

11. The liquid transfer system according to claim 10, wherein
the cartridge further comprises a passage switcher configured to switch a connection state of the first container with the first passage and a connection state of the second passage with the second container, and
the processing circuitry switches from a state in which the first container and the first passage are connected to each other to a state in which the first passage, the second passage, and the second container are connected to one another by controlling the passage switcher based on the liquid having flown into the first passage from the inside of the first container.

12. The liquid transfer system according to claim 9, wherein the cartridge further comprises a passage switcher configured to switch a connection state of the first container with the first passage and a connection state of the second passage with the second container.

13. The liquid transfer system according to claim 9, wherein the cartridge further comprises a second air filter inside the cartridge with respect to the air filter.

14. The liquid transfer system according to claim 9, wherein the cartridge further comprises a pressure chamber configured to receive depressurization and pressurization from the outside of the cartridge.

15. The liquid transfer system according to claim 14, wherein the pressure chamber has a volume larger than a possible total amount of the liquid used for the cell preparation or the cell processing.

16. The liquid transfer system according to claim 14, wherein
the pressure chamber includes a connection port communicating with the first passage, and
the connection port is positioned above a liquid surface of the liquid, the liquid surface of the liquid being observed in a state where all the liquid stored in the first container is stored in the pressure chamber.

17. The liquid transfer system according to claim 9, wherein the cartridge further comprises a storage provided for the first passage and configured to store the liquid that has flown into the first passage.

18. The liquid transfer system according to claim 9, wherein
the first container is a reagent container, a blood bag, or a culture container, and
the second container is a culture container, a collecting container, or a waste liquid container.

19. A method for moving the liquid stored in the first container to the second container by using the liquid transfer system according to claim 9, the method comprising:
causing the liquid to flow into the first passage from the inside of the first container by depressurizing the first passage; and
causing the liquid that has flown into the first passage to flow into the second container through the second passage by pressurizing the first passage.
